(19) United States
(12) Patent Application Publication
(10) Pub. No.: US 2002/0086309 A1
(43) Pub. Date: Jul. 4, 2002

(54) Device for identifying the presence of a nucleotide sequence in a DNA sample

(76) Inventors: James Benn, Arlington, MA (US); Jean-Francois Manchec, Natick, MA (US)

Correspondence Address: LAHIVE & COCKFIELD, 28 STATE STREET, BOSTON, MA, 02109, US

(21) Appl. No.: 09/939,011

(22) Filed: Aug. 24, 2001

Related U.S. Application Data
(60) Provisional application No. 60/228,239, filed on Aug. 25, 2000.
(60) Provisional application No. 60/266,035, filed on Feb. 2, 2001.
(60) Provisional application No. 60/131,660, filed on Apr. 29, 1999.
(60) Provisional application No. 60/155,299, filed on Sep. 21, 1999.

(51) Int. Cl. C12Q 1/68; C12M 1/34

(52) U.S. Cl. 435/6; 435/287.2

(57) ABSTRACT

A system and method for identifying the presence of specific nucleotide sequences in a target DNA sample is provided. The nucleotide detection system comprises a flat plate detection cell having a sample chamber, a membrane and an optical window providing optical access to the interior of the cell. A target DNA sample is mixed with labeled nucleotides and other chemistries, and undergoes a chemical reaction, such that if the DNA sample has the nucleotide, the resulting mixture will contain labeled nucleotides that have undergone a change in molecular weight. The reacted sample is applied to the sample chamber and the membrane in the flat plate nucleotide detection system is utilized to effect the separation of the smaller molecular weight labels from the sample. After separation, the presence of the label in either the sample chamber or the filtrate chamber, or on the membrane itself, is detected through the optical window to the sample chamber or through an optical window to the filtrate chamber to determine the presence or absence of the nucleotide sequence.

### REFERENCE TO RELATED APPLICATIONS

This application claims priority to U.S. Provisional Patent Application No. 60/228,239 filed Aug. 25, 2000 and U.S. Provisional Patent Application No. 60/266,035, filed Feb. 2, 2001, the contents of which are hereby incorporated by reference. The subject matter of this application relates to U.S. Provisional Application Nos. 60/131,660, filed Apr. 29, 1999, 60/155,299, filed Sep. 21, 1999, U.S. patent application Ser. No. 09/422,677, filed Oct. 21, 1999, U.S. Continuation-in-Part application Ser. No. 09/561,764, filed Apr. 28, 2000 and U.S. Patent Application, Attny. Docket No. GEN-007CP, filed Aug. 24, 2001. The aforementioned applications, and the references cited therein, are incorporated herein by reference.

### FIELD OF THE INVENTION

The present invention relates to devices and methods for detecting the presence, absence or mutation of a particular nucleotide at a specific location on a strand of DNA.

A single nucleotide position on a strand of DNA may be responsible for a polymorphism or an allelic variation. There are known disease states that are caused by such variations at a single nucleotide position. The usefulness of detecting such variations includes but is not limited to, genotyping, DNA family planning, diagnostics (including infectious disease), prenatal testing, paternal determination, pharmacogenetics, and forensic analysis.

Laboratory automation has played a key role in the advancement of genomics and drug discovery over the past decade. Automated systems are now used in high-throughput sample preparation for DNA sequencing at large sequencing centers.

Modern laboratories employ partially automated procedures for handling samples. In these procedures, reagents and templates are combined by manually feeding 96-channel pipettors with thermocycling plates.

The techniques of dialysis and ultrafiltration, although well established, are typically difficult to perform on small sample volumes without suffering loss of the sample. A significant drawback in standard 5-10 μl sequencing reactions is that at least 50% of the sample is wasted. Furthermore, the amount of fluorescently labeled DNA that can be detected on current fluorescent readers is much lower than the amounts that are typically processed. Generally, 0.5-1 μl samples are sufficient to detect fluorescently labeled DNA.

Current systems for detecting a nucleotide sequence in a DNA sample require separate stations for processing the sample, filtering the sample and detecting the presence or absence of the nucleotide sequence. Transfer of the sample between the separate stations is necessary, which adds significant time and complication to the detection process.

### SUMMARY OF THE INVENTION

The present invention provides a flat plate nucleotide detection cell for detecting the presence, absence or mutation of a nucleotide sequence in a target DNA sample. The flat plate nucleotide detection cell has a sample chamber, a membrane provided along a portion of said sample chamber for effecting separation of a sample in the sample chamber and an optical detection window providing optical access to the sample chamber or another chamber in the detection cell.

The flat plate nucleotide detection cell is used to detect a single nucleotide polymorphism (SNP) in a strand of DNA. A target DNA sample is mixed with labeled nucleotides and other chemistries, and undergoes a chemical reaction. If the DNA sample has the SNP, the resulting mixture will contain labeled nucleotides that have undergone a change in molecular weight. The illustrative flat plate nucleotide detection system may be utilized with any suitable technique for detecting a nucleotide sequence in a DNA sample. The membrane in the flat plate system is utilized to effect the separation of the smaller molecular weight labels from the sample. After separation, the presence of the label in either the sample chamber or the filtrate chamber, or on the membrane itself, is detected to determine the presence or absence of the nucleotide sequence. The flat plate system of the invention provides one or more optical detection windows to allow direct detection of a label in an interior chamber of the flat plate, without necessitating transfer of the sample to a separate detection system.

According to one aspect, a flat plate nucleotide detection cell is provided. The flat plate nucleotide detection cell comprises an upper flat plate, at least one sample chamber formed along a bottom surface of the upper flat plate, a membrane provided along a portion of the sample chamber, and an optical window. The optical window is provided in the upper channel plate, and permits light to pass between the sample chamber and a detector.

According to another aspect, a flat plate nucleotide detection cell is provided comprising an upper flat plate, a sample chamber, a membrane, a lower flat plate forming a filtrate chamber, and an optical window provided in the lower channel plate. The optical window permits light to pass between the filtrate chamber and a detector.

According to yet another aspect, a system for detecting the presence of a nucleotide sequence in a DNA sample is provided. The system comprises a flat plate detection cell having an interior chamber, a membrane provided along a portion of the interior chamber and an optical window providing access to the interior chamber. The system further includes an optical detector positioned relative to the optical window to monitor the interior chamber.

According to another aspect, a method of detecting the presence of a nucleotide sequence in a DNA sample using the flat plate detection cell is provided. The method comprises providing a flat plate detection cell, injecting an admixture containing a DNA sample into the flat plate detection cell to effect separation of the admixture and monitoring either/or a sample chamber or a filtrate chamber for the presence of a label through an optical window in the flat plate detection cell.

### BRIEF DESCRIPTION OF THE DRAWINGS

The foregoing and other objects, features and advantages of the invention will be apparent from the following description and apparent from the accompanying drawings, in which like reference characters refer to the same parts throughout the different views. The drawings illustrate principles of the invention and, although not to scale, may if necessary show relative dimensions.

FIG. 1 is a cross-sectional view of a flat plate detection system of a first embodiment of the invention, with an optical detection window for detecting the sample chamber.

FIG. 2 is a cross-sectional view of a flat plate detection system of a second embodiment of the invention, with an optical window for detecting the dialysate chamber.

FIG. 3 is a cross-sectional view of a flat plate detection system of a third embodiment of the invention with a plurality of optical windows for detecting the sample chamber and the dialysate chamber.

FIG. 4 provides dialysis results for two samples using a flat dialysis plate.

FIG. 5 is a bottom view of a dialysis chamber of the embodiments of FIGS. 1, 2 and 3;

FIG. 6 is an exploded perspective view of a fourth embodiment of the invention;

FIG. 7 is a view of a top surface of a needle guide of the fourth embodiment of the invention;

FIG. 8 is a cross-sectional view of a portion of the needle guide illustrated in FIG. 7;

FIG. 9 illustrates a bottom surface of the needle guide illustrated in FIG. 7;

FIG. 10 shows a top surface of an upper channel plate according to the fourth embodiment of the invention;

FIG. 11 shows a cross-sectional view of a portion of the upper channel plate illustrated in FIG. 10;

FIG. 12 provides a bottom surface view of the upper channel plate illustrated in FIG. 10;

FIG. 13 is an upper surface view of a lower channel plate according to the fourth embodiment of the invention;

FIG. 14 is a detailed view of a portion of the upper surface of the lower channel plate illustrated in FIG. 13;

FIG. 15 provides a bottom surface view of the lower channel plate illustrated in FIG. 13 according to the fourth embodiment of the invention;

FIG. 16 provides an upper surface view of a manifold according to the fourth embodiment of the invention; and

FIG. 17 provides a bottom surface view of the manifold illustrated in FIG. 16.

### DETAILED DESCRIPTION OF THE INVENTION

Before further description of the invention, certain terms employed in the specification, examples and appended claims are, for convenience, defined below.

The term “biological sample” refers to a sample comprising one or more cellular or extracellular components of a biological organism. Such components include, but are not limited to, nucleotides (e.g., DNA, RNA, fragments thereof and plasmids), peptides (e.g., structural proteins and fragments thereof, enzymes, etc.), and carbohydrates, etc. The biological samples described herein may also include transport media, biological buffers and other reagents well know in the art for carrying out the processes described above. Although the methods of the invention can be carried out with a biological sample of just about any volume, biological samples in accordance with the invention typically have microliter (μL) volumes and therefore can be referred to as microsamples, e.g., biological microsamples. The methods of the invention are advantageously practiced with biological samples having volumes ranging between about 10 μl and about 0.05 μL, and preferably between about 0.1 μL and about 3 μL.

The term “dialysis” is art-recognized and is understood to refer to the separation or filtering of substances in solution by means of their unequal diffusion through a membrane, including the following forms of dialysis. As used herein, “equilibrium dialysis” refers to dialysis which occurs without exchange or flow of dialysate, e.g. dialysis solution. “Flow dialysis ” refers to dialysis which occurs with a flow (or counterflow) of dialysate. “Exchange dialysis” refers to dialysis which includes at least one change of the dialysate surrounding the membrane.

The term “membrane” as used herein refers to both dialysis membranes and ultrafiltration membranes, as appropriate, to accomplish dialysis or ultrafiltration. The membrane is a material of any suitable composition and size which may used to separate or filter substances in solution by means of unequal diffusion, e.g., by size exclusion. Although dialysis membranes and ultrafiltration membranes typically are semipermeable, the term “membrane” as used herein is not so limited. Dialysis membranes and ultrafiltration membranes are closely related and are interchangeable as used herein. In most applications, ultrafiltration membranes are generally designed to withstand elevated pressures.

The term “purification” is intended to encompass, in its various grammatical forms and synonyms (e.g., purification, purifying, clean up, etc.) any operation whereby an undesired component(s) is/are separated or filtered from a desired component(s). Such operations include, but are not limited to, filtration, ultrafiltration, dialysis/equilibrium dialysis, chromatography, and the like. In certain embodiments, purification is achieved by molecular size discrimination among the components of the biological sample. Purification by molecular size discrimination can be achieved using any number of materials of varying porosity well known in the art including, but not limited to, filters, membranes, and semipermeable ultrafiltration filter materials.

The terms “sequence” refers to one or more nucleotides in a target DNA sample.

The terms “temperature processing,” “temperature treating,” and “thermal processing” are used interchangeably herein to refer to the application of a variety of temperature conditions to the sample, depending on the particular process underway and include, but are not limited to, continuous and discontinuous heating regimens, e.g., denaturation, annealing, incubation, precipitation, and the like. For example, the terms broadly encompass thermocycling associated with PCR and similar processes.

The term “ultrafiltration” refers to any method of purification, separation or filtration wherein the sample is under positive or negative pressure.

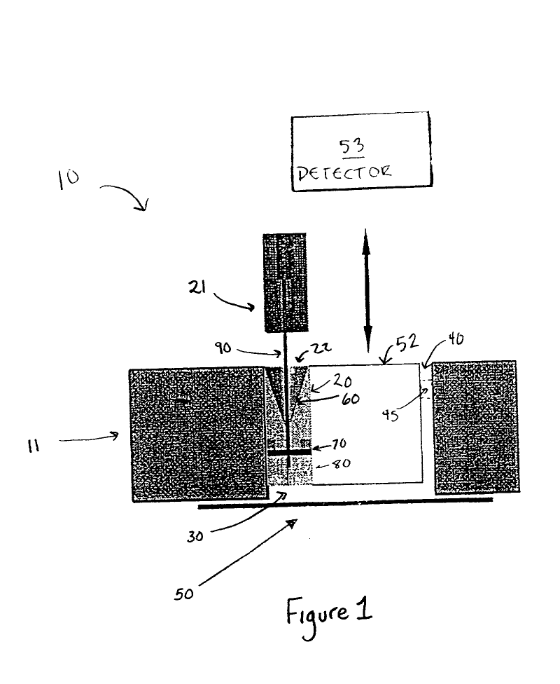

According to a first embodiment of the invention, the nucleotide detection system for detecting the presence or absence of a sequence in a target DNA sample comprises a flat plate detection cell 10, as shown in FIG. 1. The flat plate detection cell 10 comprises a top flat plate 11 including a syringe docking port 20 fluidly coupled with a sample chamber 30 for holding a sample and a vent hole 40. The syringe docking port 20 is used to direct a sample into the sample chamber 30. The vent hole 40 provides a vent for the sample chamber 30. A dialysis or ultrafiltration membrane 50 for separating a sample by means of size exclusion is provided along a portion of the sample chamber 30.

The flat plate detection cell 10 of FIG. 1 further includes an optically transparent portion in the flat plate 11 forming an optical window 52 to the sample chamber 30. The optical window 52 allows viewing and detection of a sample or other contents of the sample chamber. A detector 53, such as a fluorescent reader, may be disposed relative to the optical window 52 to detect the contents of the sample chamber 30 through the optical window 52. In this manner, the contents of the sample chamber can be monitored and detected directly, at any time during processing of a sample, without necessitating transfer of the sample to a separate detection system.

According to the illustrative embodiment, the flat plate detection cell 10 is generally opaque, excluding the optical window, which is substantially transparent. The optical window 52 may be formed of any suitable material for optically connecting an enclosed chamber within the flat plate detection cell 10 with a detector, such as styrene, polycarbonate or any material that is substantially transparent to selected wavelengths of light. According to one embodiment, the upper flat plate 11 is comprised entirely of an optically transparent material, and the opacity of the non-window portions of the flat plate is provided using paint or masks to block light. The applications of the flat plate detection cell 10 and the optical window 52 to nucleotide detection will be described in detail below.

The syringe docking port 20 preferably includes a needle guide 60, a seal 70 and a needle stop 80. Those of ordinary skill will recognize that the docking port 20 can comprise greater or fewer components, and can have any suitable size and shape. The syringe docking port 20 includes an entry portion 22 opposite a distal end. The optional needle guide 60 defines an insertion axis 90 for guiding a syringe holding a sample through the syringe docking port 20, which is preferably perpendicular to the membrane 50. The needle guide 60, formed near entry portion 22, is preferably funnel shaped so as to guide a syringe along a path intersecting the insertion axis 90. The needle guide 60 is preferably formed of polyethylene. However, other non-reactive materials may be used to form the needle guide 60.

The optional seal 70 is preferably formed to provide a fluid-tight seal within the syringe docking port 20. The seal 70 is designed to be repeatedly pierced by a syringe 21 while maintaining the ability to provide a fluid-tight seal. The seal 70 is preferably pierced upon manufacture. Alternatively, the seal 70 may be manufactured without piercing and later pierced by a sharp needle during use. The seal 70 may be formed of silicone, rubber, silicone rubber, or other elastic material, although silicone rubber is preferred.

The optional needle stop 80 can be formed near the distal end of the syringe docking port 20 to prevent a needle from piercing the membrane 50, preferably by preventing the needle from entering the sample chamber 30. As with the needle guide 60, the needle stop 80 is formed of a non-reactive material, such as polyethylene.

The flat plate detection cell 10 includes the sample chamber 30 formed in the upper flat plate 11. The sample chamber 30 is preferably formed with the distal end of the syringe docking port 20 fluidly coupled to one end of the sample chamber 30. An optional vent hole 40 is also fluidly coupled to the sample chamber 30, preferably near an opposite end of the sample chamber 30. An optional seal 45 or valve may be provided in or in fluid communication with the vent hole 40 to provide for the control of pressure within the sample chamber 30. The dialysis/ultrafiltration membrane 50 is preferably provided along a portion of the sample chamber 30. According to an alternate embodiment, the vent hole is eliminated from the flat plate detection cell 10.

The flat plate detection cell 10 of the invention may be used to separate biological samples less than one microliter by the use of the membrane 50. The membrane 50 is selected to have a molecular cutoff to retain molecules of interest and allow unwanted molecules to pass through the membrane, out of the sample, by means of dialysis or ultrafiltration. To separate a biological sample using dialysis, a dialysis solution is provided on the opposite side of the membrane 50 from the sample chamber 30. The dialysis solution may be stationary or may have a flow. To separate a biological sample using ultrafiltration, a positive or a negative pressure differential is applied to the sample chamber 30 to force the sample through the membrane 50.

The flat plate detection cells 10 of the illustrative embodiment is utilized to detect a single nucleotide polymorphism (SNP) in a target DNA sample. A target DNA sample is mixed with labeled nucleotides and other chemistries, and undergoes a chemical reaction. If the DNA sample has the SNP, the resulting mixture will contain labeled nucleotides that have undergone a change in molecular weight. A needle containing the resulting mixture of the DNA sample and the labeled nucleotides is introduced into the syringe docking port 20. The needle guide 60 guides the needle onto insertion axis 90 and into seal 70. The needle stop 80 prevents the needle from being inserted too far. The needle introduces the sample into the sample chamber 30 preferably through needle stop 80. The vent hole 40 allows for the escape of air from the sample chamber 30 as the sample is introduced. To effect dialysis of the sample, the portion of the sample chamber 30 having the membrane 50 is exposed to a dialysis solution. The membrane 50 in the flat plate system is utilized to effect the separation of the smaller molecular weight labels from the sample. After separation, the sample chamber 30 may be monitored through the optical windows 52 to detect the presence of a retained label in the sample chamber, using a detector 53. The presence of the label in the sample chamber, or on the membrane itself, indicates the presence or absence of the SNP, depending on the particular assay used. Upon completion of the separation and detection, the needle previously used to insert the sample, or a different needle, removes the sample from the sample chamber through needle stop 80. The needle may optionally be removed from syringe docking port 20 during dialysis and reinserted upon completion of dialysis to effect removal of the sample.

As discussed, the sample may also be separated using ultrafiltration, wherein a pressure differential is applied to the sample in the sample chamber 30 to effect separation of the sample.

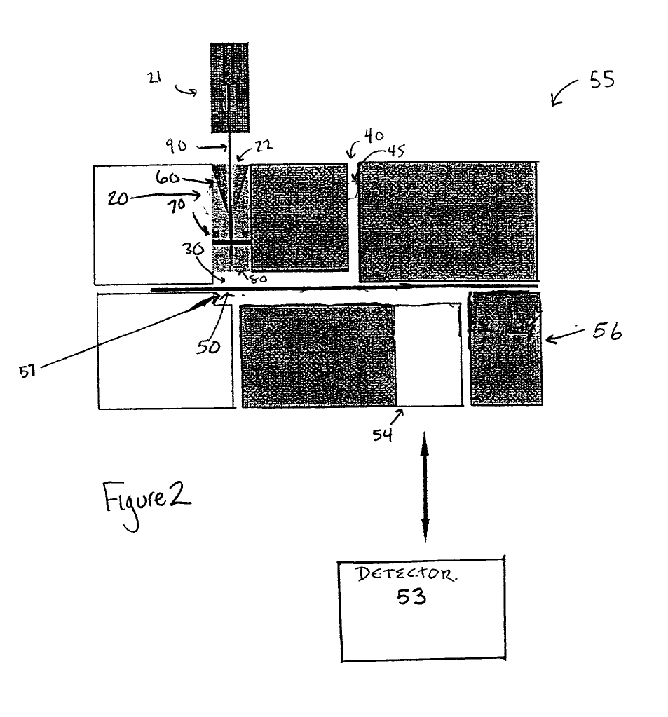

A second embodiment of the flat plate nucleotide detection system is illustrated in FIG. 2. The flat plate nucleotide detection cell 55 of FIG. 2 includes a lower plate 56 including a filtrate chamber 57 below the membrane 50 for collecting the filtrate that passes through the membrane 50. The lower plate 56 further includes an optical detection window 54 to facilitate direct detection of the filtrate in the filtrate chamber 57 without necessitating transfer of the filtrate to a separate detection system. A detector 53, such as a fluorescent reader may be disposed adjacent to the optical detection window 54 to monitor the filtrate chamber 57 and detect the presence of a label in the filtrate and identify the presence of a SNP in a target DNA sample. According to the illustrative embodiment, the membrane 50 is substantially transparent. Therefore, in order to detect the only filtrate, the filtrate chamber 57 and lower optical window 54 are shifted relative to the sample chamber 30. In this manner, only the filtrate side of the membrane will be detected through the optical window 54 and the sample side will be blocked from view. As illustrated, the optical window 54 is located in a portion of the filtrate chamber 57 that is does not overlap with the sample chamber 30. The invention is not limited a filtrate chamber that is offset from a sample chamber and it is within the scope of the invention to align the filtrate chamber with the sample chamber.

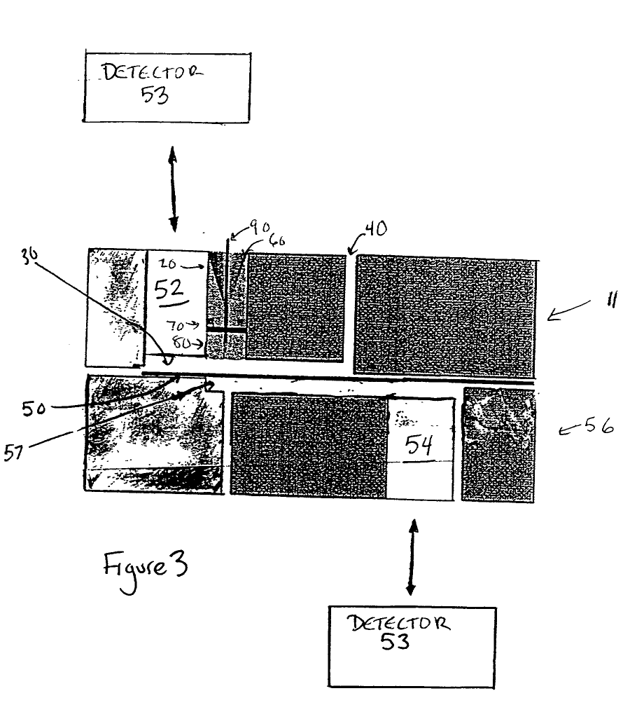

FIG. 3 illustrates a third embodiment of the flat plate nucleotide detection cell 58, including optical windows 52, 54 in both the top flat plate 10 and the bottom flat plate 56 to facilitate detection of both the sample chamber 30 and the filtrate chamber 57 using detectors 53 disposed relative to each optical window 52, 54, to detect the presence of a label in either or both chambers. As shown, the sample chamber 30 and filtrate chamber 57 are shifted relative to each other. In this manner, the contents of the sample chamber 30 can be detected independent of the contents of the filtrate chamber 57 and vice versa. One skilled in the art will recognize that the invention is not limited to the illustrated configuration, and that the optical windows 52, 54 may be positioned in any location suitable for providing an optical connection to an interior chamber formed by one or more of the flat plates 10, 56.

The illustrative flat plate nucleotide detection system may be utilized with any suitable technique for detecting a nucleotide sequence in a DNA sample. To detect a nucleotide sequence in a DNA sample, according to one application of the invention, the sample chamber 30 is loaded with a labeled probe mixture, and a target DNA sample. According to one embodiment, the sample chamber 30 is pre-loaded with labeled probe and the target DNA sample is injected into the pre-loaded sample chamber 30 through the syringe docking port 20. The chamber 30 is then temperature-cycled between approximately 30 and 95 deg C. to effect extension of the primers, and change the molecular weight of the probe. The mixture is then brought into contact with the membrane 50, which is used to separate, by means of size, those labeled species that have changed molecular weight and those that have not changed molecular weight.

For some assays, a positive identification of a DNA sequence will result in an increase in molecular weight. In this case, extended primers will be trapped in the sample chamber 30 and will not be able to pass through the membrane 50, which is selected to have a molecular cutoff to retain the extended primers having an increased molecular weight. Either dialysis or pressure driven ultrafiltration is used to drive the labeled species that did not increase in molecular weight across the membrane 50. In the ultrafiltration approach, excess water can be injected, under pressure, into the sample chamber 30, to exit through the membrane 50, carrying with it those labeled species that did not increase in molecular weight, leaving trapped on the membrane 50 those species that did increase in molecular weight. The membrane 50 is selected to have a molecular cutoff to allow passage of the lower-weight labeled species. The presence of the increased molecular-weight species is then directly detected by means of optical imaging and fluorescence detection of the chamber and/or the membrane using the optical detection window 52 in the device illustrated in FIGS. 1 and 3. The sample can be detected directly and immediately after separation, or at any time during the sample processing, without necessitating transfer of the sample to a separate detection system.

For other nucleotide detection assays, a positive identification of a DNA sequence will result in a decrease in molecular weight of the labeled primer or nucleotide. In this case, the labeled primer or nucleotide passes through the membrane 50, which is selected to have a molecular cutoff to allow passage of the labeled primer or nucleotide, and is detected on the opposite side of the membrane 50 from the original sample through an optical window 54 located on the lower flat plate 57. Either dialysis or ultrafiltration is used to drive these species across the membrane 50.

The illustrative embodiment may be used in conjunction with any suitable technique for detecting a single nucleotide polymorphism (SNP) within a DNA molecule, such as the exonuclease assay described in U.S. Pat. No. 5,391,480, the contents of which are incorporated herein by reference, wherein a labeled primer extends during thermocycling, thus increasing in molecular weight. Other suitable assays for use with the illustrative embodiment involve the single base extension of a primer by adding a labeled nucleotide upon match of a single nucleotide polymorphism (SNP) site, disclosed in U.S. Pat. Nos. 5,888,819 and 6,004,744. The contents of both patents are incorporated herein by reference.

According to another application, the flat plate detection system is used with a SNP assay as described in U.S. application Ser. No. 60/266,035, the contents of which are incorporated herein by reference. Briefly, the SNP assay described in U.S. application Ser. No. 60/266,035 provides a method for detecting the presence or absence of a first nucleotide, at a position within a DNA molecule in a sample by forming an admixture of a primer and a strand of DNA of the sample and imposing conditions such that a hybridization product is formed between the primer and the DNA strand. The primer comprises a sequence of DNA, which hybridizes with the strand of DNA adjacent to the first nucleotide position and has a second nucleotide opposite the first nucleotide position. The second nucleotide has an associated label (e.g., a fluorescent label, a radioactive label or a mass-tag) and hybridizes to the first nucleotide in the event that the second nucleotide is complementary to the first nucleotide. The second nucleotide does not hybridize to the first nucleotide in the event that the second nucleotide is not complementary. A proofreading polymerase is applied to the hybridization product under conditions in which the second nucleotide is preferentially excised to form a labeled nucleotide product in the event that the second nucleotide is not hybridized to the first nucleotide, and in which the second nucleotide is preferentially incorporated into a primer extension product in the event that the second nucleotide is hybridized to the first nucleotide.

The presence or absence of a label in excised nucleotides and extension products may then be detected using the flat plate detection system of the illustrative embodiment. The admixture is injected, e.g., from a syringe 21, through the syringe docking port 20, into the sample chamber 30 of the flat plate detection cell 10 and into contact with a first side of the membrane 50. The membrane 50 of the corresponding flat plate detection cell is selected to have a molecular weight cut-off such that the labeled nucleotide excision product may pass through (or passes through quickly), the primer may not pass through (or passes through slowly), and the extension product may not pass through. For a dialysis separation, a dialysis solution is applied to a second side of the membrane opposite the first side of the membrane to effect separation of the components. Alternatively, a pressure is applied to the sample to effect separation of the components through ultrafiltration. The filtrate chamber 57 in the flat plate detection cell 10 may then be directly observed through the optical detection window 54 to determine the presence of the label in the filtrate. Any suitable detection means may be utilized, including direct fluorescence measurement, or mass spectrometry. The sample on the first side of the membrane 50 may also be monitored through an optical detection window 52 in the upper flat plate 11 for the presence of a label after providing sufficient time for separation of the various components of the sample to occur. The presence of a label in the filtrate chamber 57 in concentrations greater than a background amount after a first predetermined time period (nucleotide excision product) is indicative of the absence of the first nucleotide, and the presence of a label remaining in the sample chamber 30 in concentrations greater than a background amount after a second predetermined time period that is greater than the first predetermined time period (extension product) is indicative of the presence of the first nucleotide.

Detection methods well known to those skilled in the art may be employed to determine the presence or absence of a label in the extension product through one or more of the optical windows 52, 54. For example, the reaction products are fractionated by the membrane 50 to separate excised nucleotides, primers, and extension products. These components (or mixtures thereof) are then independently tested for the presence or absence of a label. According to the illustrative embodiment, the second nucleotide in an assay used to detect the presence or absence of a particular nucleotide has a fluorescent label and the presence or absence of the fluorescent label in excised nucleotides and extension products is detected by direct fluorescence or by using fluorescence polarization through one or more of the optical windows 52, 54. When a fluorescent sample is exposed to polarized light at its absorption wavelength, fluorophores of appropriate transition moment orientation are excited. The fluorescent light emitted from such molecules is polarized like the incident light, but the polarization decreases by the extent to which the molecules have rotated during the time between absorbing and emitting light. Consequently, the decrease in polarization measures the rotation of the molecules during the lifetime of the excited state. In the situation where a fluorescent label is retained during the synthesis of the extension product, the extension product containing the label will undergo a slower rotational Brownian motion because of its higher effective volume/mass and an increase in the fluorescence polarization will be observed. In the situation where the fluorescent label is excised prior to the synthesis of the extension product, the free fluorescent label will undergo a faster rotational Brownian motion because of its lower effective volume/mass and a decrease in the fluorescence polarization will be observed.

The fluorescence polarization measurements may be performed using any suitable instruments available in the art including the FluoroMax-2 instrument (available from Instruments S. A., Edison, N.J.), FP777 spectrofluorimeter equipped with a microcomputer-assisted polarization measurement module and a Peltier temperature regulation system (available from Jasco, Tokyo), and the Analyst HT microplate reader (available from Molecular Devices Corp.).

According to alternate embodiments, a radioactive label is used and the detector 53 comprises a radiometric detector for detecting the presence or absence of the radioactive label in the sample chamber 30 or the filtrate chamber 57. One skilled in the art will recognize that a variety of types of labels and corresponding detectors may be utilized with the present invention.

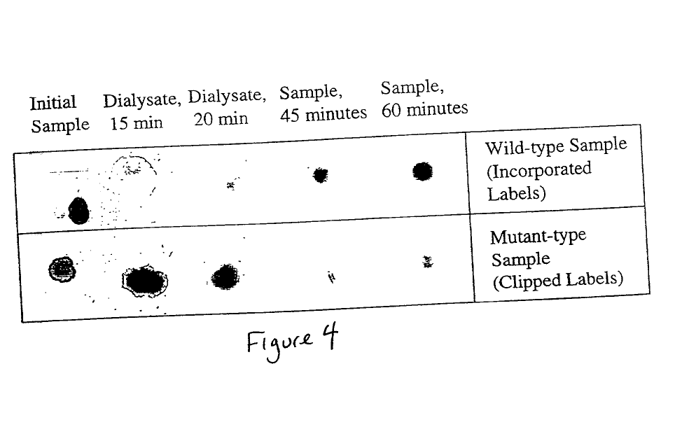

FIG. 4 illustrates dialysis results of a DNA sample that is dialyzed and detected using a flat dialysis plate, such as that described in copending U.S. application Attny. Docket No. GEN-007CP, filed Aug. 24, 2001. In the experiment of FIG. 4, a labeled primer was used to make extension products from wild-type DNA and mutant DNA carrying a single base substitution at a single site. After PCR, five microliters of the wild type sample were placed in a first flat dialysis plate, and five microliters of the mutant type sample were placed into another flat dialysis plate. Both flat dialysis plates contained Spectrum Brand 100k MWCO CE Dialysis membranes. The dialysis side of each of the plates was loaded with five microliters of distilled water. In one experimental run, after 15 minutes and after 20 minutes, the water from the dialysis side was removed and transferred to two glass slides, respectively. In another sample run, after 45 minutes and after 60 minutes, the sample was removed and transferred to two glass slides, respectively. A Fuji fluorescent reader was used to perform the detection of the sample and the dialysate on each of the glass slides.

The top row of FIG. 4 illustrates the results from the wild-type sample. The bottom row of FIG. 4 illustrates the results from the mutant-type sample. The first column shows an initial reading of the sample before any dialysis has taken place, indicating that the labels were present on the sample side of the membrane in both the wild (label incorporated) and mutant (label clipped) samples. The second and third columns show the results of detection of the dialysis solution after 15 and 20 minutes of dialysis, respectively. Where the labels were incorporated in the extension product of the wild-type samples, no clipped labels were present to dialyze across the membrane, thus none were detected by the fluorescent reader. Where the labels were clipped due to the presence of a mutation, they diffused across the membrane from the sample into the dialysis solution, and were detected by the fluorescent reader.

The fourth and fifth columns show the original samples after dialysis for 45 and 60 minutes. During the PCR process, extension products were made from the labeled primers. Where the labels were retained on the primers, they were incorporated into the extension products, and where they were clipped off by the exonuclease reaction, they were not incorporated. During the long dialysis time, the primers that did not participate in the reaction, and the single labeled nucleotides, were all substantially dialyzed out of the sample, resulting in only extension products remaining in the sample chamber. Fluorescence was detected in the extension products that retained the labels, and no fluorescence was detected in the extension products that did not retain the labels. Therefore, the ability to differentiate between the wild type (incorporated labels) from the mutant type (clipped labels) was demonstrated in two ways. The first way was by detecting labeled nucleotides in the dialysis solution, where clipping had taken place, and the second was by detecting incorporated labels in the dialyzed sample solution, where no clipping had taken place.

The same experiment as described above can be more rapidly and efficiently performed in a flat plate dialysis system of the invention, such as that illustrated in FIG. 3. The built in optical windows 52 and 54 enable detection without the need for transferring the sample and/or dialysate to another device for detection.

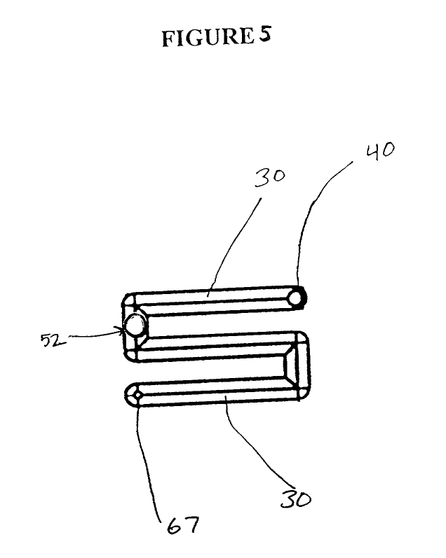

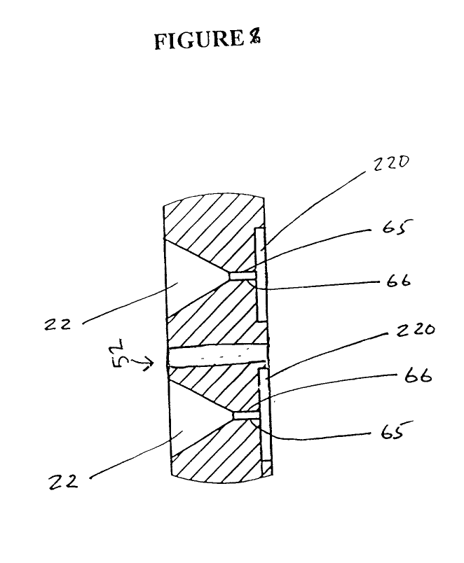

FIG. 5 illustrates a top view of the sample chamber 30. The sample chamber 30 shown in FIG. 5 is preferably formed with a diameter of less than 1 mm and greater than 0.1 mm, preferably having a volume of less than 1 microliter. A diameter of approximately 0.5 is preferred. The sample chamber is preferably formed in the shape of an elongated tube cut along its longitudinal axis, thereby forming a flat portion along substantially all its length. The sample chamber may be formed in a serpentine shape, such as an S shape as is shown in FIG. 5, or may be straight. The sample chamber 30 shown in FIG. 5 shows a lower portion 67 of a guide channel 65 (shown in FIG. 8) in fluid communication with the sample chamber 30, near an end of the sample chamber 30. A vent hole 40 is also illustrated in fluid communication near an opposite end of the sample chamber 30. An optical window 52 may be located along any point of the sample chamber to allow for detection of the sample in the sample chamber.

Optional washing of the flat plate detection cell 10, or any part thereof, may be performed after separation of the sample and detection of the sample chamber and/or filtrate chamber. Preferably, an alcohol-based solution is used. Washing may be performed with or without disassembly of the flat plate detection cell 10.

The flat plate detection cell 10 is easily optionally multiplied into an array of multiple flat plate detection cells, each having a corresponding optical window or a set of optical windows, allowing each flat plate detection cell 10 to use a portion of a single, continuous membrane 50.

The invention is capable of processing and detecting many samples in parallel, if desired, using standard micro-titer plates as reagent sources. The system can be used to retrieve, mix and dispense fluids by integration with air or liquid-filled volumetric devices, such as piezoelectric elements, movable pistons or syringe-type plungers.

A syringe needle docking system comprising at least one syringe 21, may be used to automate the insertion and removal of samples. The syringe needle docking system may optionally include automated syringe needle movement and automated syringe plunger actuation.

The dimensions of the sample chamber 30 provide for the use of small sample volumes while providing a large surface area for the sample to be in contact with the membrane 50. It is desirable to maximize the surface area of the sample chamber 30 along the membrane 50 for a given sample chamber 30 volume. However, the surface tension of the sample is an important consideration to allow for the maximum recovery of a sample from the sample chamber by a needle through the needle stop 80. Preferably, the sample chamber 30 diameter is between about 1.0 mm and about 0.1 mm. Specifically, approximately about 0.5 mm is preferred. A large surface area along the membrane 50 allows for more rapid separation of a sample. This large surface area is provided without need for additional components, such as those disclosed in U.S. Pat. No. 5,679,310 to Manns.

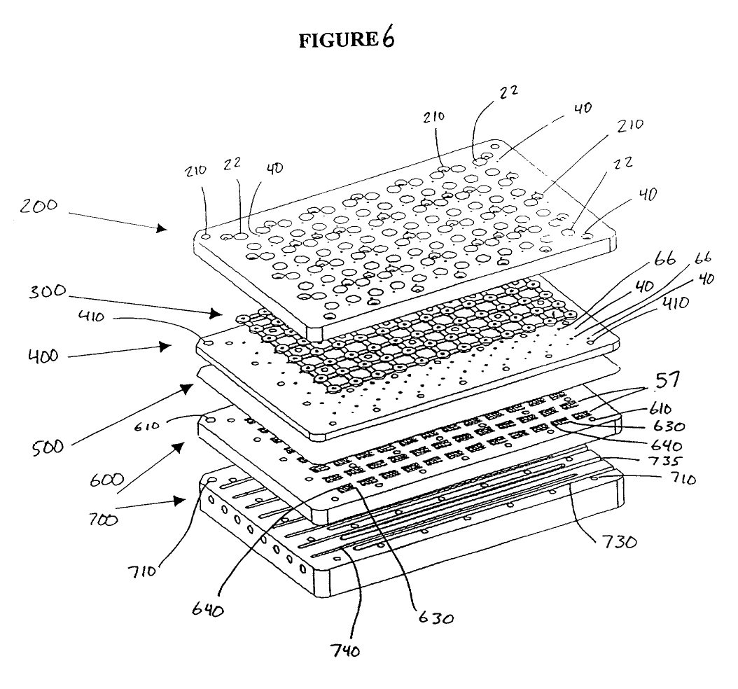

Another embodiment of the invention is shown in FIG. 6. The flat plate detection system 100 shown in FIG. 6 preferably includes a needle guide 200, a seal 300, an upper channel plate 400, a membrane 500, a lower channel plate 600 and a manifold 700. Preferably, a plurality, such as 96 or 384 or more, flat plate detection cells are provided in the flat plate detection system 100, as described below. Those of ordinary skill will recognize that any suitable number of cells can be employed. According to the illustrative embodiment, a plurality of the flat plate detection cells in the flat plate detection system 100 include one or more optical windows to allow direct detection of the sample chamber and/or a filtrate chamber. One skilled in the art will recognize that the invention is not limited to the illustrative embodiment and that any number of flat plate detection cells may be provided with one or more optical windows to provide access to the sample chamber 30 and/or the filtrate chamber 57 of a selected number of flat plate detection cells in the flat plate detection system 100.

A needle guide 200 is provided with a plurality of holes. For each flat plate detection cell 10, an entry portion 22 and a vent hole 40 are preferably provided within needle guide 200. For each flat plate detection cell 10 having an optical window 52 to the sample chamber, an optical window 52 is preferably provided within the needle guide as well. Alignment holes 210 are also preferably provided to aid in mounting of the various components of the flat plate dialysis system 100 to each other.

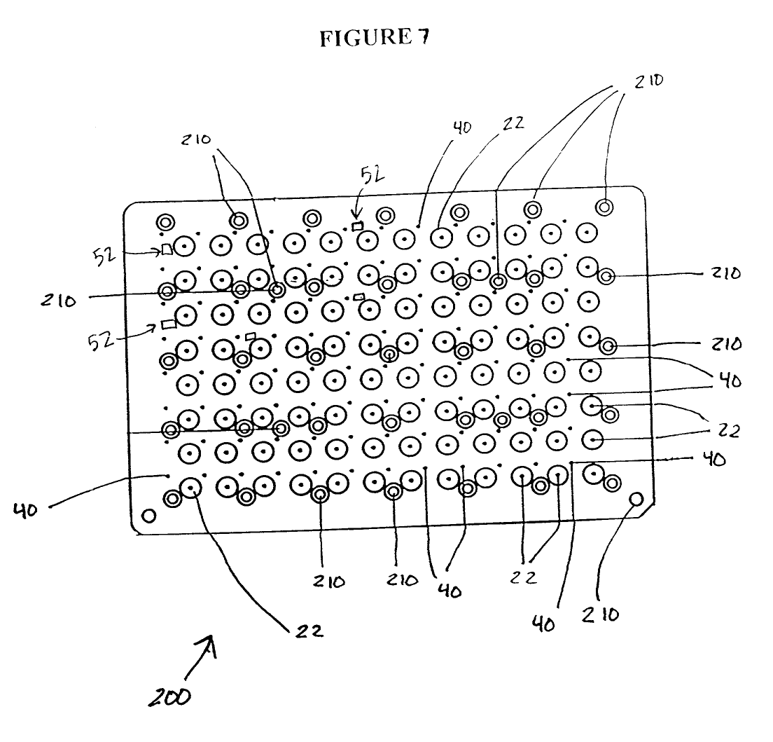

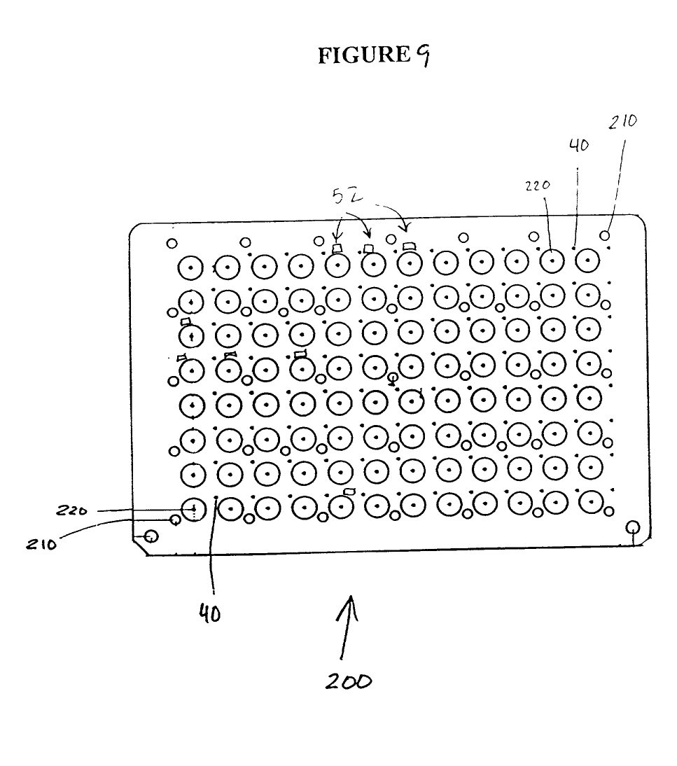

A cross-section of the needle guide 200 shown in FIG. 7 is provided in FIG. 8. Entry portions 22 are fluidly coupled via an upper portion 66 of the guide channel 65, preferably to an annular seal receiving portion 220. As shown in FIG. 9, the bottom surface of needle guide 200 preferably provides an annular seal receiving portion 220 for each flat plate detection cell 10. FIG. 9 also illustrates a vent hole 40 corresponding to each annular seal receiving portion 220.

As shown in FIG. 6, the optional seal 300 is preferably provided between the needle guide 200 and the upper channel plate 400. The seal 300 is preferably configured so as to mate with the annular seal receiving portion 220 to provide a fluid-tight seal along the guide channel 65.

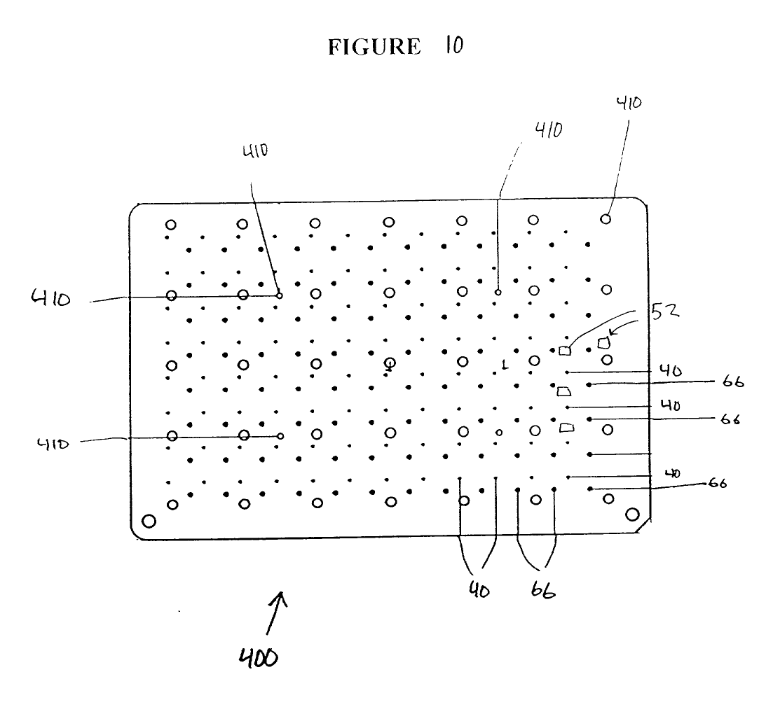

The upper channel plate 400 is described with reference to FIG. 10. FIG. 10 illustrates a pattern of holes similar to those provided in the needle guide 200 in that a pair of two holes is provided for each flat plate detection cell 10. However, the upper channel plate 400 differs from the needle guide 200 in that the upper channel plate 400 preferably provides a needle stop, analogous to needle stop 80 of the first embodiment of the invention. An upper portion 66 of the guide channel 65 corresponding to a flat plate detection cell 10 is shown in FIG. 7. A corresponding vent hole 40 is also provided, as shown in FIG. 7. Optical windows 52 are provided in the upper channel plate 400 at selected locations to facilitate detection of a corresponding sample chamber.

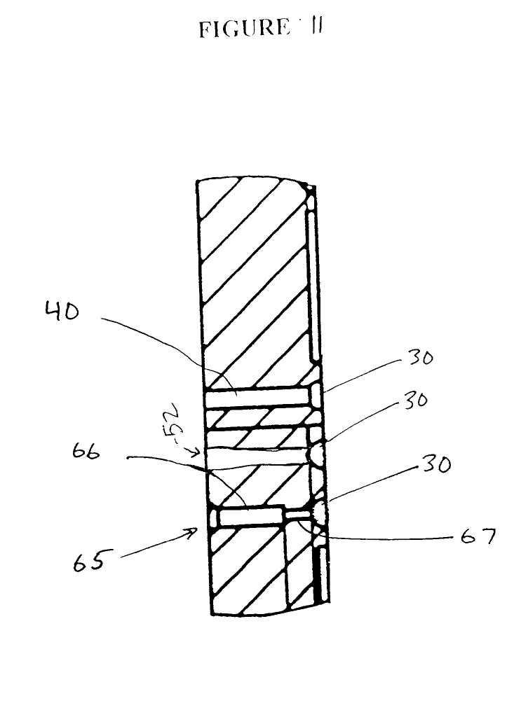

A cross-section of a portion of the upper channel plate 400 is provided in FIG. 11. A guide channel 65 is shown having an upper portion 66 and a lower portion 67. The lower portion 67 of the guide channel 65 preferably has a needle stop formed by a reduced diameter so as to prevent a needle from traveling within the lower portion 67 of the guide channel 65. A vent hole 40 is also provided within the upper channel plate 400. The vent hole 40 may be provided with a varying diameter. An optical window 52 is also provided in the upper channel plate 400. FIG. 11 also illustrates a cross-section of the sample chamber 30 in fluid communication with the lower portion 67 of the guide channel 65 and the vent hole 40 and in optical communication with the optical window 52.

It is within the scope of the invention to provide an optional seal valve in or in fluid communication with the vent hole 40. Such a seal may be provided to facilitate elevated or reduced pressure within the sample chamber 30.

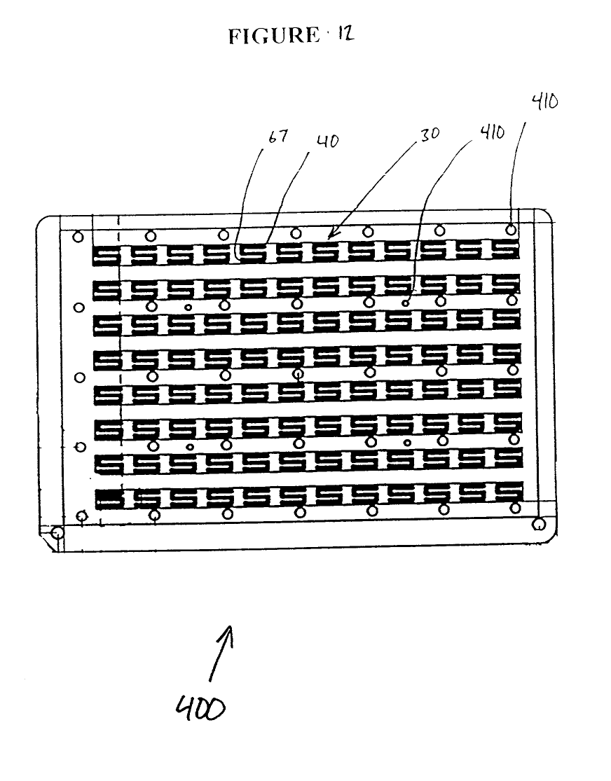

FIG. 12 illustrates a bottom surface of the upper channel plate 400. A sample chamber 30 is provided for each flat plate detection cell 10. A vent hole 40, optical window 52 and a lower portion 67 of the guide channel 65 are illustrated in FIG. 12 and correspond to those shown in FIG. 10. Alignment holes 410 are preferably provided within the upper channel plate 400 to correspond to the alignment holes 210 of the needle guide 200.

It is within the scope of the invention to integrally form the needle guide 200 and the upper channel plate 400 in a unitary piece.

As shown in FIG. 6, the membrane 500 is provided between the upper channel plate 400 and the lower channel plate 600. The membrane 500 may optionally be bonded to upper channel plate 400 or may be mounted by a compressive force applied to keep the upper channel plate 400 and the lower channel plate 600 together. Bonding may be performed by ultrasonic welding, heat bonding or a variety of adhesives.

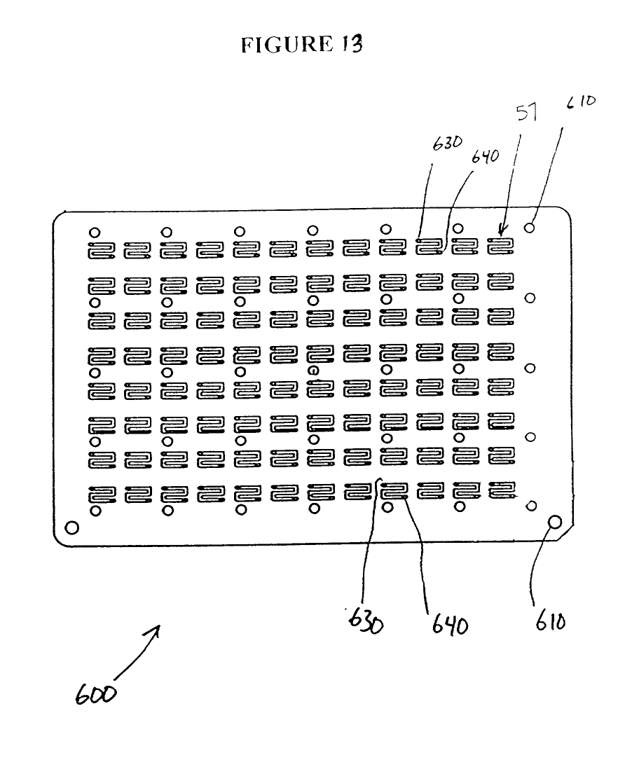

As shown in FIG. 13, optionally, an upper surface of the lower transfer plate 600 provides filtrate chambers 57 to correspond to the sample chambers 30 of the upper channel plate 400 shown in FIG. 9. As discussed, the filtrate chambers 57 are generally offset from the sample chambers 30 to facilitate detection of either or both chambers through one or more of the optical windows 52, 54.

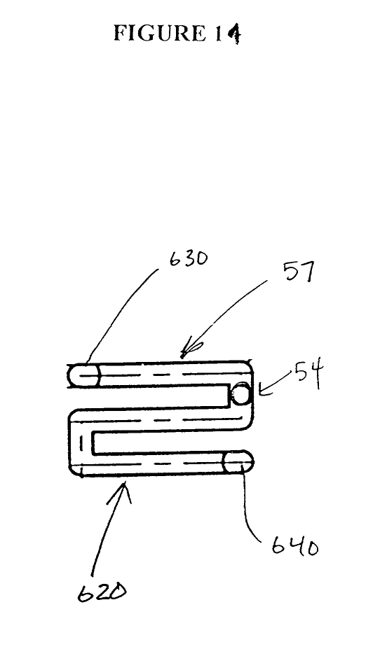

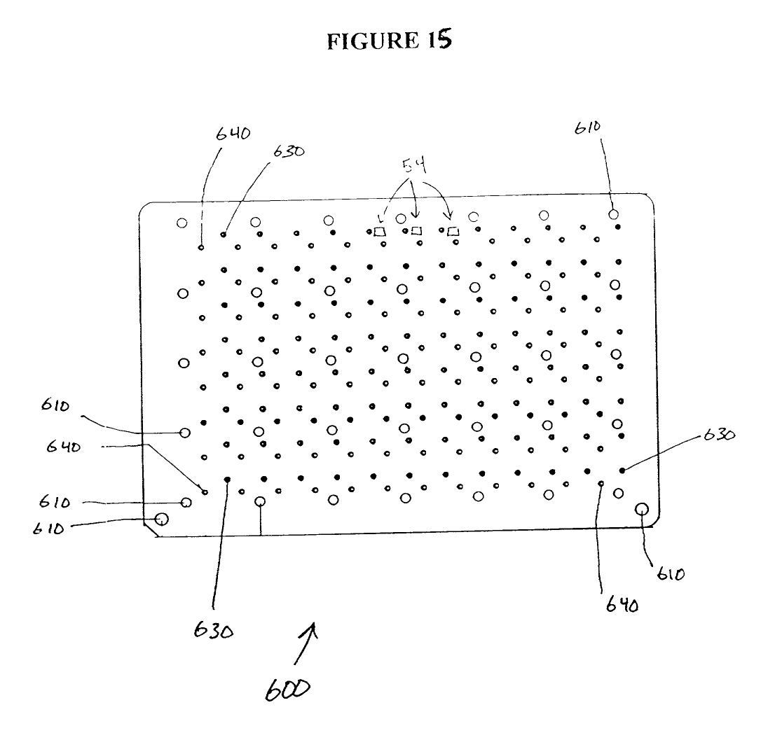

Each filtrate chamber 57 may be provided with a first port 630 and a second port 640. An optical window 54 may also be provided along the filtrate chamber 57. A detailed view of the filtrate chamber 57 is provided in FIG. 14. FIG. 15 shows a bottom surface view of the lower channel plate 600. Alignment holes 610 are optionally provided within the lower channel plate 600 to correspond to the alignment holes in other components of the flat plate dialysis system 100.

It is within the scope of the scope of the invention to provide an optional seal or valve within or in fluid communication with the first port 630 and/or second port 640 to aid in altering a pressure within the filtrate chamber 57 or sealing the filtrate chamber.

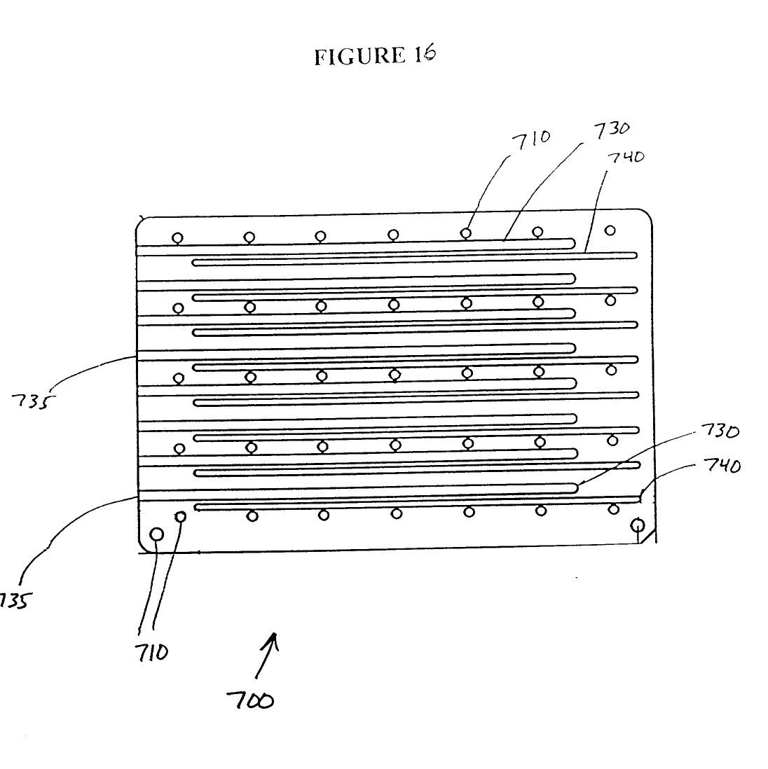

Optionally, a manifold 700 is provided under the lower channel plate 600. Manifold 700, as shown in FIG. 16, provides on an upper surface, a first and a second trough 730, 740. First and second troughs 730, 740, are fluidly coupled to first and second ports 630, 640, respectively, of the lower channel plate 600. First trough 730 fluidly communicates with a first external port 735. Second trough 740 preferably does not communicate with an external port. Both first and second troughs 730, 740 allow fluid communication among first and second ports 630, 640 along a row of flat plate dialysis cells 10 within the flat plate dialysis system 100. Optionally, alignment holes 710 are provided within the manifold 700 to correspond to alignment holes of the other components of the flat plate detection system 100.

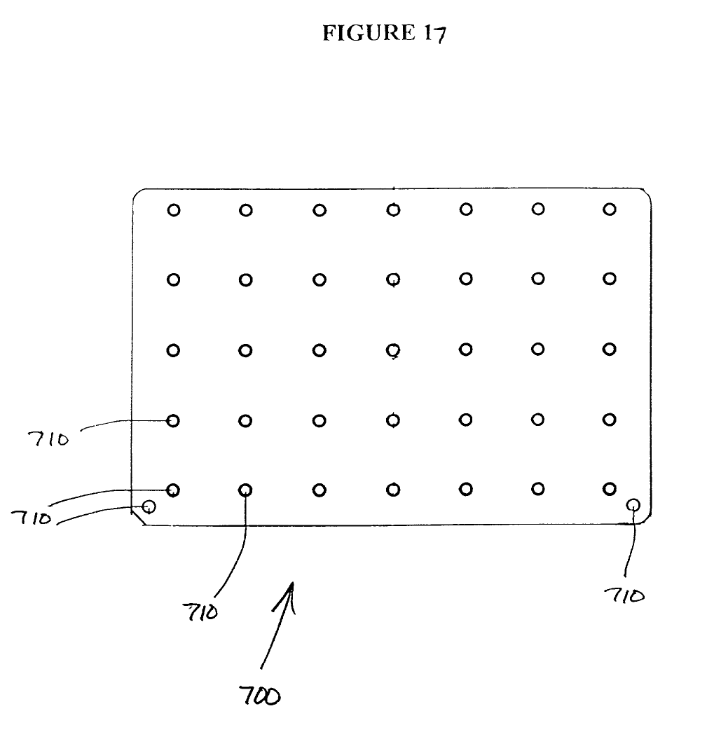

FIG. 17 provides a view of a bottom surface of the manifold 700. The lower channel plate 600 and the manifold 700 are both optional components of the flat plate dialysis system 100. Processing of a sample, such as conducting dialysis or thermocycling, can be performed in the dialysis chamber 30 by passing a dialysis solution along the dialysis membrane 500 with or without the filtrate chamber 57 of the lower channel plate 600.

In operation, the flat plate detection system 100 is adapted to be used with a syringe needle docking system or a multi-channel pipettor system, such as a 96 or 384 or more channel pipettor. Pipettor syringes are provided to align with the entry portions 22 shown in FIGS. 6, 7 and 8. The needles of the pipettor syringes are inserted into the needle guide 200 each along an insertion axis 90, shown in FIG. 8. The needles travel along the guide channel 65. The guide channel 65 is provided with a larger diameter along an upper portion 66 and a narrower diameter along lower portion 67. The lower portion 67 of the guide channel 65 preferably does not allow the needle to pass within it. A fluid-tight seal is provided by the seal 300 preferably seated within the annular seal receiving portion 220, illustrated in FIGS. 3, 5 and 6.

The needles deposit a biological sample to be analyzed and the accompanying reagents necessary for effecting a reaction through the lower portion 67 of the guide channel 65 into the sample chamber 30. The sample flows freely into the sample chamber 30 due to vent hole 40 allowing the release of air contained within the sample chamber 30. As discussed above, an optional seal 45 or valve may be provided within or in fluid communication with vent hole 40 to regulate the flow through vent hole 40.

The components of the flat plate detection cell 10, 55, 58 and flat plate detection system are preferably formed of non-reactive plastic. As discussed, the optical windows 52, 54 are formed of a suitable transparent material, such as styrene or polycarbonate and the non-window portions of the flat plate detection cell are substantially opaque. Specifically, components such as the needle guide 200, upper channel plate 400, lower channel plate 600 and manifold 700 may preferably be formed of hydrophobic materials, such as polystyrene, polycarbonate, TEFLON™, or DELRIN™. Optional coatings of TEFLON™ or silane may also be used to enhance hydrophobic properties of these materials. The membrane 50, 500, may preferably be formed of cellulose, cellulose ester, TEFLON™, polysulfone and polyethersulfone. The membrane 50, 500 is selected to have a molecular cutoff suitable for separating a DNA sample mixed with labeled nucleotides.

A further variation of the invention allows the use of alignment holes 210, 410, 610 and 710 for the passage of a temperature-controlled solution so as to vary the temperature of the dialysis chamber 30 and/or filtrate chamber 57. Thermocycling may be achieved, for example, by blowing air of different temperatures, although a liquid medium could also be used for heat transfer. Alternatively, additional holes or passages may be provided to allow for the distribution of a temperature controlled fluid to effect the temperature of dialysis chamber 30 or filtrate chamber 57 and the dialysis membrane 500.

Also within the scope of the invention are various devices to hold the needle guide 200, upper channel plate 400, dialysis membrane 500, lower channel plate 600 and manifold 700 together. For example, compression bolts may be provided within the alignment holes 210, 410, 610, 710 of the invention to compress the flat plate dialysis system. Screws may also be used in place of or in combination with compression bolts. Other devices, such as C-clamps or large hose clamps may be used to hold the needle guide 200, upper channel plate 400, dialysis membrane 50, lower channel plate 600 and manifold 700 together. Any of the above-described items may also be used with a subset of components of the flat plate SNP detection system.

Another variation of the invention involves the use of a beveled corner on each of the needle guide 200, the upper channel plate 400, lower channel plate 600 and manifold 700, or any subset thereof, to aid in alignment of these components of the flat plate dialysis system, as shown in FIG. 6.

The present invention can be used with a conventional fluid dispensing unit, such as a Hydra dispenser, manufactured by Robbins Scientific. Those of ordinary skill will also recognize that other fluid dispensing and sample handling units, whether in modular or discrete forms, can be employed to work with the invention.

The present invention provides benefits over current systems for detecting sequences in DNA samples. The invention integrates the processing, separation and detection systems and processes into a single device, and prevents unnecessary transfer of the sample between separate systems. The accuracy of the detecting process is improved while the time, cost and complication involved in detecting a nucleotide sequence are significantly reduced.

The present invention has been described by way of example, and modifications and variations of the exemplary embodiments will suggest themselves to skilled artisans in this field without departing from the spirit of the invention. Features and characteristics of the above-described embodiments may be used in combination. The preferred embodiments are merely illustrative and should not be considered restrictive in any way. The scope of the invention is to be measured by the appended claims, rather than the preceding description, and all variations and equivalents that fall within the range of the claims are intended to be embraced therein.

Having described the invention, what is claimed as new and protected by Letters Patent is:

1. A flat plate nucleotide detection cell, comprising:
an upper flat plate;
a sample chamber formed along a bottom surface of said upper flat plate for holding a sample;
a membrane provided along a portion of said sample chamber for separating a sample in the sample chamber, and
an optical window provided in said upper flat plate, said optical window for permitting light to pass between the sample chamber and a detector for monitoring the sample chamber.

2. The flat plate nucleotide detection cell of claim 1, further comprising:
a syringe docking port in said upper flat plate fluidly coupled to the sample chamber.

3. The flat plate nucleotide detection cell of claim 2, said syringe docking port further comprising:
a seal for providing a fluid-tight seal after being pierced by a needle of a syringe;
a needle stop for preventing the needle from entering said sample chamber.
a needle guide formed in a funnel shape in said syringe docking port to guide the needle toward said sample chamber.

4. The flat plate nucleotide detection cell of claim 2, wherein said syringe docking port further comprises a seal capable of providing a fluid-tight seal after being pierced by a needle.

5. The flat plate nucleotide detection cell of claim 2, wherein said syringe docking port further comprises a needle stop capable of preventing a needle of a syringe from entering said sample chamber.

6. The flat plate nucleotide detection cell of claim 2, further comprising a needle guide formed in said syringe docking port to guide a needle of a syringe toward said sample chamber.

7. The flat plate nucleotide detection cell of claim 6, wherein said needle guide is funnel shaped.

8. The flat plate nucleotide detection cell of claim 1, further comprising a vent hole in fluid communication with the sample chamber providing a vent for the sample chamber.

9. The flat plate nucleotide detection cell of claim 1, wherein the membrane comprises a flat sheet.

10. The flat plate nucleotide detection cell of claim 1, further comprising a filtrate chamber mated to said sample chamber via said membrane.

11. The flat plate nucleotide detection cell of claim 1, further comprising a lower flat plate coupled to the upper flat plate and forming a filtrate chamber, wherein the membrane is mounted between the lower flat plate and the upper flat plate, such that the sample chamber and the filtrate chamber are separated by the membrane.

12. The flat plate nucleotide detection cell of claim 11, wherein the lower flat plate includes a second optical window capable of transmitting light between the filtrate chamber and a detector for monitoring the filtrate chamber.

13. The flat plate nucleotide detection cell of claim 12, wherein the filtrate chamber is offset from the sample chamber.

14. The flat plate nucleotide detection cell of claim 1, wherein the sample chamber is serpentine.

15. The flat plate nucleotide detection cell of claim 14, wherein the sample chamber is S-shaped.

16. The flat plate nucleotide detection cell of claim 1, wherein the membrane has a molecular cut-off such that a labeled nucleotide excision product passes through the membrane.

17. A flat plate nucleotide detection cell, comprising
an upper flat plate;
at least one sample chamber formed along a bottom surface of said upper flat plate;
a lower flat plate forming a filtrate chamber,
a membrane for separating a sample provided along a portion of said sample chamber and mounted between the upper channel plate and the lower channel plate such that the sample chamber and the filtrate chamber at least partially overlap; and
an optical window provided in said lower channel plate, said optical window for permitting light to pass between the filtrate chamber and a detector for monitoring the filtrate chamber.

18. The flat plate nucleotide detection cell of claim 17, further comprising:
a syringe docking port in said upper flat plate fluidly coupled to the sample chamber.

19. The flat plate nucleotide detection cell of claim 18, wherein said syringe docking port further comprises:
a seal for providing a fluid-tight seal after being pierced by a needle of a syringe;
a needle stop for preventing the needle of the syringe from entering said sample chamber.
a needle guide formed in a funnel shape in said syringe docking port to guide the needle of the syringe toward said sample chamber.

20. The flat plate nucleotide detection cell of claim 18, wherein said syringe docking port further comprises a seal for providing a fluid-tight seal after being pierced by a needle.

21. The flat plate nucleotide detection cell of claim 18, wherein said syringe docking port further comprises a needle stop for preventing a needle from entering said sample chamber.

22. The flat plate nucleotide detection cell of claim 18, further comprising a needle guide formed in said syringe docking port to guide a needle toward said sample chamber.

23. The flat plate nucleotide detection cell of claim 22, wherein said needle guide is funnel shaped.

24. The flat plate nucleotide detection cell of claim 17, further comprising a vent hole in fluid communication with the sample chamber providing a vent for the sample chamber.

25. The flat plate nucleotide detection cell of claim 17, wherein the membrane comprises a flat sheet.

26. The flat plate nucleotide detection cell of claim 17, wherein the filtrate chamber is offset from the sample chamber.

27. The flat plate nucleotide detection cell of claim 17, wherein the sample chamber is serpentine.

28. The flat plate nucleotide detection cell of claim 27, wherein the sample chamber is S-shaped.

29. A system for detecting the presence of a nucleotide sequence in a DNA sample, comprising:
a flat plate detection cell having an interior chamber, a membrane provided along a portion of the interior chamber and an optical window providing access to the interior chamber; and
an optical detector positioned relative to the optical window to monitor the interior chamber.

30. The system of claim 29, wherein the interior chamber comprises a sample chamber holding a biological sample, and the optical detector monitors the biological sample.

31. The system of claim 29, wherein the interior chamber comprises a filtrate chamber for collecting a filtrate from the membrane, and the optical detector monitors the filtrate.

32. A method of detecting the presence of a nucleotide sequence in a DNA sample, comprising,
providing a flat plate detection cell having a sample chamber, a filtrate chamber, a membrane provided between the sample chamber and the filtrate chamber and having a molecular weight cut-off such that a labeled nucleotide excision product passes through the membrane and an optical window providing access to one of the sample chamber and the filtrate chamber;
injecting an admixture into said sample chamber and into contact with a first side of said membrane, said admixture comprising a hybridization product formed of a primer and a strand of DNA in said sample, wherein the primer comprises a sequence of DNA which hybridizes with said strand of DNA adjacent to said first nucleotide position and having a second nucleotide opposite said first nucleotide position, said second nucleotide associated with a label, said second nucleotide hybridizing to said first nucleotide in the event said second nucleotide is complementary to said first nucleotide and said second nucleotide not hybridizing to said first nucleotide in the event said second nucleotide is not complementary, and wherein a proofreading polymerase has been applied to the hybridization product under conditions in which said second nucleotide is preferentially excised to form a labeled nucleotide excision product in the event said second nucleotide is not hybridized to said first nucleotide, and in which said second nucleotide is preferentially incorporated into an extension product in the event said second nucleotide is hybridized to said first nucleotide;
applying one of a dialysis solution to the second side of the membrane and a pressure differential to the sample chamber along the first side of the membrane to pass a labeled nucleotide excision product through the membrane; and
monitoring at least one of the group of: the sample on the first side of the ultrafiltration membrane and a filtrate on the second side of the ultrafiltration membrane, for the presence of a label through said optical window, wherein the presence of a label in the filtrate in concentrations greater than a background amount after a first predetermined time period is indicative of the absence of the first nucleotide, and the presence of a label remaining in the ultrafiltration chamber in concentrations greater than a background amount after a second predetermined time period greater than said first predetermined time period is indicative of the presence of the first nucleotide.
